(19) United States
(12) Patent Application Publication
(10) Pub. No.: US 2020/0054004 A1
(43) Pub. Date: Feb. 20, 2020

(54) METHOD FOR PREPARING A TRANSPLANT

(71) Applicant: Fraunhofer-Gesellschaft zur Förderung der angewandten Forschung e.V., Munich (DE)

(72) Inventors: Christiane Wetzel, Dresden (DE); Jessy Schönfelder, Dresden (DE); Simona Walker, Dresden (DE); Jörg Kubusch, Dresden (DE)

(73) Assignee: Fraunhofer-Gesellschaft zur Förderung der angewandten Forschung e.V., Munich (DE)

(21) Appl. No.: 16/345,580

(22) PCT Filed: Nov. 15, 2017
(86) PCT No.: PCT/EP2017/079364

§ 371 Date: Apr. 26, 2019

(30) Foreign Application Priority Data
Nov. 16, 2016 (DE) 10 2016 121 982.7

(51) Int. Cl. A01N 1/02; A61L 2/10; A61L 27/36

(52) U.S. Cl. CPC A01N 1/0294; A01N 1/021; A61L 2430/40; A61L 27/3604; A61L 27/3691; A61L 2/10

(57) ABSTRACT

A method for preparing a transplant obtained from animal or human tissue is provided. The transplant is first deposited in a first liquid that comprises at least one substance that initiates and/or activates collagen cross-linking. After removing the transplant from the first liquid, the transplant, now moistened with said first liquid, is exposed to ultraviolet radiation and then to accelerated low-energy electrons.

### CROSS-REFERENCE TO RELATED APPLICATIONS

This application is a 371 nationalization of international patent application PCT/EP2017/079364 filed Nov. 15, 2017, which claims priority under 35 USC § 119 to German patent application 10 2016 121 982.7 filed Nov. 16, 2016. The entire contents of each of the above-identified applications are hereby incorporated by reference.

### DETAILED DESCRIPTION

In human medicine, transplants of animal or human origin are increasingly used to extend the life span of people and/or increase their quality of life. A challenge in the preparation of such transplants prior to the use as transplants in a recipient is to combine a sterilization of the tissue to be transplanted while preserving biofunctionality and biocompatibility. Transplants are considered biocompatible if they do not negatively interfere with the metabolism when coming into direct contact with living tissues. This affects all transplantable tissues of human or animal origin. At this point, heart valves, fascias, meninges, tendons, ligaments, skin, blood vessels, bones, and cornea are mentioned here.

Collagen-based transplants are subject to degradation by matrix metalloproteases, such as described, for example, in Goo; Hwang; Choi; Cho; Suh; Development of collagenase-resistant collagen and its interaction with adult human dermal fibroblasts, Biomaterials 24, 2003, pp. 5099-5113. The degradation rate can be influenced by the targeted use of networking agents. In Arcidiacono; Corvi; Severiist, Functional analysis of bioprosthetic heart valves, Journal of Biomechanics 38, 2005, pp. 1483-1490, it is disclosed that pericardial tissue is linked to chemical pathways through the use of toxic glutaraldehyde and then can be used as a material for biological heart valve prostheses. In the process, glutaraldehyde interlinking also reduces the antigenicity of the tissue, increases mechanical stability, and works in a disinfecting manner. Despite these advantages, there are also publications that refer to accelerated calcification of the material through the glutaraldehyde networking (Gendler; Nimni, Toxic reactions evoked by glutaraldehydes-fixed pericardium and cardiac valve tissue bioprosthesis, Journal of Biomedical Materials Research, Vol. 18, 1984, pp. 727-736). The functional duration of the valves is severely restricted by calcification and degradation. The average period until failure of a glutaraldehyde crosslinked valve prosthesis ranges from 10 to 15 years, depending on the age of a patient.

The physical modification of transplant tissue is known as an alternative to chemical crosslinking. For example, a networking of collagen is achieved by dehydrothermal treatment in a vacuum furnace at 110° C. for 3 to 5 days (Weadock; Olson; Silver, Evaluation of Collagen Crosslinking Techniques, Biomaterials, Medical Devices, and Artificial Organs, Vol. 11, No. 4, 1983, pp. 293-318). Advantages of this method are given in that they do not cause cytotoxic side effects and cause a sufficiently high tensile strength of the treated collagen. On the other hand, the long-term treatment duration of a few days, as well as denaturization and degradation of the collagen, is detrimental.

Another networking method is the treatment of collagen fibers with UV radiation, which usually only causes a superficially occurring modification, Weadock, Miller, Bellincampi, Zawadsky, Dunn, Physical crosslinking of collagen fibres: Comparison of ultraviolet irradiation and dehydrothermal treatment, Journal of Biomedical Materials Research, Vol. 29, 1995, pp. 1373-1379. On the other hand, it is disadvantageous that this procedure also leads to degradation.

It is also known to modify collagen-containing materials using gamma rays. In contrast to UV rays, gamma rays have a very high range (da Silva Aquino, Sterilization by Gamma Irradiation, Gamma Radiation, 2012, pp. 171-206) and thus generally irradiate the entire substrate including packaging. In addition to a desired networking of the collagen and a sterilization of the substrate, this process is accompanied by strong degradation and denaturation phenomena in the material.

Finally, In U.S. Pat. No. 6 203 755 B1, sterilization of biological tissues with high-energy electron radiation is described. In doing so, accelerated electrons with energy in the MeV range are used. In this procedure, changes in the configuration of the collagen polypeptide chains are disadvantageous.

The invention is therefore based upon the technical challenge of creating a method for preparing transplants of animal or human origin, by which the disadvantages of the prior art can be overcome. In particular, it should be possible with the method according to the invention to yield a good networking of the collagen in transplants and the sterilization of transplants without the use of toxic substances and yet preserve the biocompatibility of the transplants.

In the method according to the invention for preparation of a transplant obtained from animal or human tissue, the transplant is first deposited in a first liquid, wherein at least one substance is contained in the first fluid, which initiates and/or activates the networking of collagen. Such substances can include, for example, an enzyme, an amino acid, and/or a sugar. It is particularly advantageous for the method according to the invention if a photoinitiator is used as a substance that initiates and/or activates the networking of collagen. A photo-initiator is a chemical compound that decomposes after absorption of ultraviolet light in a photolysis reaction and forms reactive species, which in turn can initiate or activate a reaction. For example, a vitamin, such as riboflavin, can be used as photo-initiator.

The depositing of the transplant in the first liquid, in which the transplant is to absorb the first fluid and the photo-initiator contained therein, can range in its time frame from several minutes to several days, depending on the type of transplant. What time span is appropriate for a specific task can be determined with laboratory tests.

After the time for the transplant getting soaked with the first liquid has elapsed, the transplant is removed from the first liquid and, still moist from the first liquid, exposed, if possible, over the entire surface of the transplant, to electromagnetic radiation at the wave length range from 310 nm to 450 nm, i.e. ultraviolet radiation, in order to achieve networking of the collagen in the transplant. The substance contained in the first liquid, which was absorbed by the transplant when immersed in the first fluid, works in a supporting manner, here. The full-surface exposure of the transplant with ultraviolet light (UV) can, for example, be realized by the transplant being irradiated overall and simultaneously with several UV radiation sources, or by means of the various surface areas of the transplant being exposed successively with ultraviolet radiation using one or more UV radiation sources. In one embodiment of the method according to the invention, the exposure of the transplant with ultraviolet radiation is performed with a dose from 100 mJ/cm2 to 2500 mJ/cm2.

The method according to the invention also comprises the processing step of exposing the entire surface of the transplant to accelerated, low-energy electrons, which is carried out after exposing the transplant to ultraviolet radiation. By exposing the transplant to accelerated, low-energy electrons, the transplant is sterilized, on the one hand, and the impingement with accelerated, low-energy electrons causes an increase in the networking level of the collagen in the transplant, on the other hand.

In another embodiment, between the exposure to ultraviolet radiation and the impingement with accelerated, low-energy electrons, the transplant is immersed in a second liquid. This is particularly advantageous if the time between exposure of the substrate with ultraviolet radiation and the impingement of the substrate with accelerated, low-energy electrons is a greater time period provided with the drying of the transplant. Here, it is advantageous if a substance is also contained in the second liquid which initiates and/or activates the crosslinking of collagen and this way promotes the further crosslinking of the collagen in the transplant during the impingement with accelerated, low-energy electrons.

In another option, the transplant is impinged only with accelerated, low-energy electrons after the transplant has been inserted into a packaging, which, for example, is made from plastic, metal, or may also be made of glass.

The present invention is explained in greater detail below with reference to an exemplary embodiment. The pericardial tissue of a pig shall be prepared as a transplant. For this purpose, residual tissue is first removed of the pericardial tissue using processing steps of prior art and all cellular components of the pericardial tissue are removed. After this treatment, any remaining pericardial tissue is referred to below as the transplant.

According to the invention, the transplant is immersed for a period of 20 hours in a riboflavin solution with a concentration of 260 μmol/L, which contains 2% dextran T500 in order for the transplant to be soaked with the photo-initiator riboflavin. If riboflavin is used as a photo-initiator in the method according to the invention, riboflavin solutions with a concentration from 0.1 μmol/L to 300 mmol/L are suitable as the first liquid, for example. Then the transplant is removed from the riboflavin solution and the planar transplant, still moist with the riboflavin solution, is exposed at both sides for 30 minutes each to ultraviolet radiation with a power density of 0.3 mW/cm2 in order to cause networking of the collagen in the transplant.

Another crosslinking of the collagen in the transplant and the sterilization of the transplant is done according to the invention by impinging the transplant at both sides with accelerated, low-energy electrons after treatment with UV radiation. In order to maintain the biofunctionality of the transplant, only low-energy electrons are used in the method according to the invention. In this process, accelerated electrons with an energy from 100 keV to 20 500 keV are used and the impingement of a transplant with low-energy electrons occurs according to the invention with a dose from 1 kGy to 500 kGy.

In the exemplary embodiment, atmospheric pressure is applied in the processing chamber and the air in the processing chamber is enriched with nitrogen until an oxygen ratio of maximally 100 ppm is adjusted. Alternatively, when impinging a transplant with accelerated, low-energy electrons, other pressure ratios may also be adjusted in the processing chamber and other gases or gas mixtures can also be introduced into the processing chamber. Suitable for the method according to the invention are, for example, air, helium, argon, carbon dioxide, carbon monoxide, nitrogen monoxide, oxygen, neon, methane, krypton, hydrogen, or mixtures of at least two previously named components.

The method according to the invention was described using pericardial tissue as an example, but it is not limited to this type of tissue. Rather, the method according to the invention is suitable for all transplantable types of tissues of animal and human origin. Here, heart valve, fascia, meninges, tendon, ligature, skin, blood vessel, bone, and cornea tissues are listed.

In the case of application of the method according to the invention on transplant tissues of various types of tissue, the experimental evidence could be provided that a very good networking of the collagen in the transplant and the sterilization of the transplant can be achieved even without the use of toxic substances and while preserving biocompatibility. In transplant tissue prepared in accordance with the invention only minor or no calcification at all of the tissue could be detected, since the calcification-promoting substance glutaraldehyde is not used in the method according to the invention. Therefore, a longer shelf life of the transplants can be assumed.

To clarify the use of and to hereby provide notice to the public, the phrases “at least one of <A>, <B>, . . . and <N>” or “at least one of <A>, <B>, <N>, or combinations thereof” or “<A>, <B>, . . . and/or <N>” are defined by the Applicant in the broadest sense, superseding any other implied definitions hereinbefore or hereinafter unless expressly asserted by the Applicant to the contrary, to mean one or more elements selected from the group comprising A, B, . . . and N. In other words, the phrases mean any combination of one or more of the elements A, B, . . . or N including any one element alone or the one element in combination with one or more of the other elements which may also include, in combination, additional elements not listed. Unless otherwise indicated or the context suggests otherwise, as used herein, “a” or “an” means “at least one” or “one or more.”

1. A method for preparing a transplant that was obtained from animal or human tissue, the method comprising:
immersing the transplant in a first liquid containing a substance that initiates and/or activates networking of collagen;
removing the transplant from the first liquid and exposing the transplant, moistened with the first liquid, to ultraviolet radiation;
impinging the transplant with accelerated, low-energy electrons.

2. The method of claim 1, wherein an enzyme, an amino acid, and/or a sugar are included in the substance that initiates and/or activates networking of collagen.

3. The method of claim 1, wherein a photo-initiator is included in the substance that initiates and/or activates networking of collagen.

4. The method of claim 3, wherein the photo-initiator includes a vitamin.

5. The method of claim 1, wherein the exposure of the transplant to ultraviolet radiation is carried out over the entire surface of the transplant.

6. The method of claim 1, wherein the exposure of the transplant to ultraviolet radiation is carried out at a dose that is in a range from 100 mJ/cm2 to 2500 mJ/cm2.

7. The method of claim 1, wherein accelerated electrons are used with an energy that is in a range from 100 keV to 500 keV.

8. The method of claim 1, wherein the impingement of a surface area of a substrate with low-energy electrons is done with a dose that is in a range from 1 kGy to 500 kGy.

9. The method of claim 1, wherein the transplant, between the exposure to ultraviolet radiation and the impingement with accelerated, low-energy electrons, is immersed in a second liquid.

10. The method of claim 7, wherein a second liquid is used, which contains a substance that initiates and/or activates networking of collagen.
